(19) United States
(12) Patent Application Publication
(10) Pub. No.: US 2023/0080071 A1
(43) Pub. Date: Mar. 16, 2023

(54) MONITORING OF SUSTAINED BLAST PRESSURE WAVE EXPOSURE

(71) Applicant: INTERNATIONAL BUSINESS MACHINES CORPORATION, ARMONK, NY (US)

(72) Inventors: Corville O. Allen, Morrisville, NC (US); Caroline K. Cameron, Durham, NC (US)

(21) Appl. No.: 17/447,715

(22) Filed: Sep. 15, 2021

(51) Int. Cl. G08B 21/02; G08B 17/00; G16H 50/20; G06K 9/00

(52) U.S. Cl. CPC G08B 21/02; G08B 17/00; G16H 50/20; G06K 9/0063

(57) ABSTRACT

According to one embodiment, a method, computer system, and computer program product for diagnosing one or more blast injuries of an individual based on sustained blast exposure is provided. The present invention may include identifying the presence of a blast event, monitoring the location of an individual for the duration of the blast event; determining a blast exposure associated with the blast event for the individual; determining a sustained blast exposure of the individual comprising the blast exposures associated with a plurality of blast events during a single, continuous period of time; and, responsive to the sustained blast exposure of the individual exceeding one or more threshold values, prompting the individual to seek medical attention for one or more blast injuries associated with the one or more threshold values.

### BACKGROUND

The present invention relates, generally, to the field of computing, and more particularly to digital blast injury diagnosis.

A blast injury is a complex type of physical trauma resulting from direct or indirect exposure to an explosion. Blast injuries can be caused by a high-order explosive event, which may be an explosive event where explosive material burns rapidly enough to produce an overpressure wave. High-order explosive events may be caused by the detonation of high-order explosives such as mining charges or ammonium nitrate fuel oil, as well as the deflagration of low-order explosives in certain conditions, such as aerosolized gasoline or grain dust. Blast injuries are caused by blast overpressure waves or shock waves, which may cause internal damage in the human body such as to the gastrointestinal tract, the lungs, the auditory system, the brain, et cetera. While some blast injuries may be external and clearly assessed, the majority of blast injuries are characterized by an absence of external injuries and may present symptoms days or even weeks after exposure to the blast, making it extremely difficult to detect the presence and severity of blast injuries. The field of digital blast injury diagnosis is concerned with utilizing modern computing technology to accurately identify and diagnose blast injuries.

### SUMMARY

According to one embodiment, a method, computer system, and computer program product for diagnosing one or more blast injuries of an individual based on sustained blast exposure is provided. The present invention may include identifying the presence of a blast event, monitoring the location of an individual for the duration of the blast event; determining a blast exposure associated with the blast event for the individual; determining a sustained blast exposure of the individual comprising the blast exposures associated with a plurality of blast events during a single, continuous period of time; and, responsive to the sustained blast exposure of the individual exceeding one or more threshold values, prompting the individual to seek medical attention for one or more blast injuries associated with the one or more threshold values.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates an exemplary networked computer environment according to at least one embodiment;

FIG. 2 is an operational flowchart illustrating a blast exposure monitoring process according to at least one embodiment;

FIG. 3 is a block diagram of internal and external components of computers and servers depicted in FIG. 1 according to at least one embodiment;

FIG. 4 depicts a cloud computing environment according to an embodiment of the present invention; and

FIG. 5 depicts abstraction model layers according to an embodiment of the present invention.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. In the description, details of well-known features and techniques may be omitted to avoid unnecessarily obscuring the presented embodiments.

Embodiments of the present invention relate to the field of computing, and more particularly to digital blast injury diagnosis. The following described exemplary embodiments provide a system, method, and program product to, among other things, utilize an individual's location, local climate data, and blast data during one or more blast events to assess blast exposure and diagnose a blast injury. Therefore, the present embodiment has the capacity to improve the technical field of digital blast injury diagnosis by utilizing blast data readings from sensors on or proximate to the individual affected by the blast event, the location of the individual during the blast event, and local climate data; using data tailored to the blast conditions at the location of the individual, determining blast exposure by analyzing the location of the individual to determine the individual's location relative to the blast as well as terrain features, structures, and other forms of cover, and incorporating local climate data enables the present embodiment to more accurately identify the presence and severity of blast injuries for each of potentially multiple different individuals exposed to the blast event, provide information on the effect of climate data and exposure on blast injuries, and diagnose the cumulative effects of multiple blast events on a single individual.

As previously described, a blast injury is a complex type of physical trauma resulting from direct or indirect exposure to an explosion. Primary blast injuries are caused by blast overpressure waves or shock waves, which may cause internal damage in the human body such as to the gastrointestinal tract, the lungs, the auditory system, the brain, et cetera. References to blast injuries hereinafter may be understood to refer to primary blast injuries, rather than, for example, secondary blast injuries, which are caused by ballistic trauma rather than pressure. While some blast injuries may be external and clearly assessed, the majority of blast injuries are characterized by an absence of external injuries and may present symptoms days or even weeks after exposure to the blast, making it extremely difficult to detect the presence and severity of blast injuries. The field of digital blast injury diagnosis is concerned with utilizing modern computing technology to accurately identify and diagnose blast injuries.

The extent and types of primary blast-induced injuries depend not only on the peak of the overpressure but may further depend on additional parameters such as the number of overpressure peaks, the time between overpressure peaks, the characteristics of the shear fronts between overpressure peaks, frequency resonance, and electromagnetic pulse, among others, and may further depend on the cumulative effect of multiple blast events experienced by an individual. The extent and types of blast injuries may further be affected by the position of the affected individual relative to the blast events and environmental features, and local geospatial-temporal data during the blast events. As such, it may be advantageous to, among other things, implement a system that assesses a blast exposure as a function of blast data measured at or near the person of the individual during one or more blast events, the location of the individual relative to the blast and one or more environmental features, and local climate data, and diagnosing a blast injury based on the assessed blast exposure; one skilled in the art would understand such a system to more accurately identify the presence and severity of blast injuries for each of potentially multiple different individuals exposed to the blast event thanks to individually tailored blast and location data, provide information on the effect of climate data and exposure on blast injuries, diagnose the cumulative effects of multiple blast events on a single individual, and recommend screening and/or treatment for blast injuries based on level of blast exposure rather than the manifestation of visible symptoms.

According to at least one embodiment, the invention is a system to determine a blast exposure experienced by an individual during a blast event based on the location of the individual, blast data associated with the individual, and geospatial/temporal data, and diagnosing a blast injury based on the blast exposure.

According to at least one embodiment, the invention is a system to determine a sustained blast exposure experienced by an individual during one or more blast events based on the location of the individual, blast data associated with the individual, and geospatial/temporal data, and diagnosing a blast injury based on the sustained blast exposure.

In some embodiments of the invention, blast data may be data pertaining to the pressure changes produced by a blast event. A blast event may be a high-order explosive event, which is an explosive event capable of producing an overpressure wave. An overpressure wave may be a wave of pressure in excess of atmospheric pressure which propagates outwards from the site of the blast event and is often followed by a blast wind of negative pressure, which is pressure that is less than atmospheric pressure. The overpressure wave may interact with surfaces that may change the properties of the overpressure wave and/or may create multiple complex blast waves. For example, overpressure waves from blast events that occur near or within hard solid surfaces may be amplified due to shock wave reflection, causing an increase to their destructive potential; individuals located between a blast and a building may suffer more extensive blast injuries than when the blast occurs in open space. Even in open field conditions, the overpressure wave may reflect from the ground, generating reflective waves that interact with the primary wave and change its characteristics. Environmental/climate factors, such as weather, climate, atmospheric density, et cetera affect the medium through which the blast waves propagate and may influence the characteristics of the blast waves. The blast data may include all data pertaining to the unique pressure characteristics of all blast waves originating from a blast event, including the initial overpressure wave, any subsequent related overpressure waves, and/or the pressure fluctuations between overpressure waves, including negative pressure waves, experienced at the position of the individual. The blast data may be recorded by a blast sensor, which may be a pressure sensor on the person of an individual, such as in the individual's helmet, belt, integrated into a mobile device, et cetera. In some embodiments of the invention the blast sensor may be within a threshold distance of the individual. Blast data may be recorded at regular and frequent intervals, for example once a second, and may be recorded at all times.

In some embodiments of the invention, the system may identify a presence and duration of a blast event, which may be determined in real time or near-real-time based on the blast data. The system may identify the presence of a blast event responsive to receiving a pressure wave of a magnitude that exceeds a threshold value, where the threshold value represents a pressure wave of a magnitude which has been found to likely correspond with that of a blast event, and at or below which represents pressure fluctuations that are unlikely to have been caused by a blast event. In some embodiments of the invention, the system may identify the presence of a blast event where the magnitude and frequency of one or more pressure waves match a profile associated with an explosion. The system may determine when the blast event is over when, subsequent to identifying the presence of a blast event, the blast sensors record no pressure waves exceeding the threshold value for greater than a threshold duration, where the threshold duration represents a maximum amount of time that may pass without pressure waves from a blast event before the blast event can be considered over. The system may calculate the duration of the blast event by calculating the elapsed time between the system's identifying the presence of the blast event and determining that the blast event is over.

In some embodiments of the invention, location data may be the location associated with an individual; the location associated with the individual may often be the location of a mobile device that is associated with the individual and assumed to be on the person of the individual, such that its location corresponds with the location of the individual. Location data may be, for example, recorded by software agents capable of deducing the geographical location of a mobile device connected to the Internet, for example using the IP address, MAC address, et cetera. Location data may also include geolocation data of the individual or a mobile device associated with the individual gathered by sensors such as global positioning systems, microlocation data gathered via near field communications, Bluetooth, RFID tags, beacons, et cetera, and any other location data gathered for instance via cameras and motion detectors, laser rangefinders, et cetera. Such sensors may be integrated into a mobile device on the person of the individual, deployed in the environment of the individual, or some combination of the two. In some embodiments of the invention, location data may only be recorded during a blast event.

In some embodiments of the invention, geospatial/temporal data may be data used to gather the effect of climate indicators on characteristics of the blast waves such as blast intensity and radius over the duration of the blast event. The geospatial/temporal data may include any data pertaining to environmental factors or conditions of the climate in the area of the blast event at the time of the blast event that may have an effect on the propagation of pressure waves through the environment, including temperature, wind speed, wind direction, humidity, air quality, air density, rainfall, barometric pressure, et cetera. The geospatial/temporal data may be collected through one or more geospatial/temporal sensors and/or received or acquired from a database, online service, website, or other form of online or local data repository, and may be data pertaining to prevailing environmental conditions during the blast event at the location of the individual.

In some embodiments of the invention, blast exposure may be a representation of the individual's exposure to a given blast event at given intervals of time, for example every second, or every half a second, during the blast event, and may comprise all features or information of the blast, location of the individual, environmental conditions, and/or surrounding terrain features necessary to assess the individual's exposure to the blast and likelihood of sustaining a blast injury. The blast exposure may include all pressure waves experienced by the individual over the course of a blast event, including the peak of the initial pressure wave, the duration of the overpressure, the time from the incident blast wave, and the frequency of the waves. The blast exposure may further include the type of pressure waves experienced by the individual, including complex waves, reflective waves, Friedlander waves, et cetera.

In some embodiments of the invention, the system may determine the location of the blast based on the location of the individual and the blast data during the duration of the blast event. In some embodiments, the system may receive the location of the blast, for example from a system designed to triangulate or otherwise locate a blast. The blast exposure may include the individual's geographical proximity to the blast over the course of each blast event, taking into account the location and movement of the individual.

In some embodiments of the invention, the blast exposure may be based on physical features such as structures, hills, rocks, et cetera near the individual which may offer shelter from the blast. The system may analyze maps of the terrain near the individual to determine, for example using object detection methods, topographical data, geological surveys, et cetera, what physical features such as structures, geological/topographical features, objects, et cetera were near the individual. In some embodiments of the invention, the system may determine the strength, density, pressure conduction, material composition, flexibility, or other properties of one or more physical features, for example by looking up physical properties of objects identified using object detection methods, determining the type and composition of rocks and terrain features based on geological data, et cetera. In some embodiments of the invention, for example where the system identifies the location of the blast, the system may identify all or some subset of physical features between the individual and the location of the blast at any point during the duration of the blast event, such that the physical features may have shielded the individual or disrupted the pressure waves caused by the blast event prior to the pressure waves reaching the individual. The system may adjust the blast exposure based on the extent to which physical features shielded the individual from pressure waves; such adjustment may be based on the number, size, strength, and position of the physical features relative to the individual at any given interval of time within the blast event.

In some embodiments of the invention, for example where blast data and location data is being recorded for a plurality of individuals, the system may locate the blast based on the location and recorded pressure waves associated with a plurality of individuals at the same interval of time and/or during the same blast event.

In some embodiments of the invention, the blast exposure may be a single value enumerating the individual's exposure to a single blast event at given intervals of time during the blast event. The blast exposure may be expressed as a single value, and/or may be assessed at each of a number of intervals during the blast event; in some embodiments of the invention, the blast exposure may be expressed as a single value that is the weighted sum of the blast exposure values assessed at each interval during the blast event. The weighted sum may be based on the weighted sum model (WSM) in decision theory for multi-criteria decision analysis (MCDA). The value may be calculated by assigning weights to each of a number of factors drawn from the geospatial/temporal data, blast data, and/or location data, where the weight may represent the relative amount to which the factor contributes to the exposure and/or likelihood of blast injury. The weight associated with any given factor may be dynamically adjusted based on new data, to more precisely represent the amount to which the factor contributes to the likelihood of blast injury. For example, weights may be assigned to particular factors as follows, where higher weights represent higher exposure:

1 Location Factors:
a. Within a threshold distance of a blast (within threshold distance for initial wave=3; within threshold distance for multiple waves=3; within threshold distance for single wave=2; within threshold distance for no waves=0)
b. Environmental surroundings (confined space=3; near structure or building=3; open space=2)

2. Blast Data Factors:
a. Type of blast wave (complex wave=3; reflective wave=2; Friedlander wave=1)
b. Peak of initial pressure wave (high=3; moderate=2; low=1)
c. Duration of overpressure (high=3; moderate=2; minimal=1)
d. Time from incident blast wave (high=3; moderate=2; low=1)
e. Frequency of waves (high=3; moderate=2; low=1)

3. Geospatial-temporal Data Factors:
a. Temperature (high=3; moderate=2; minimal=1)
b. Wind speed (high=3; moderate=2; low=1)
c. Wind direction (towards individual=3; away from individual=2)
d. Humidity (high=3; moderate=2; low=1)
e. Air quality (poor=3; moderate=2; good=1)
f. Rainfall (high=1; moderate=1; none=2)
g. Barometric pressure (high=3; moderate=2; low=1)

The blast exposure value at an interval may be calculated by summing up the weights associated with every factor present during the blast event. For example, for a blast event where the individual was within a threshold distance from blast during multiple pressure waves (weight =3), was out in the open (weight=2), the duration of the overpressure was minimal (weight=1), the frequency of the waves was low (weight=1), and the weather was hot (weight=3) with high winds (weight=3), low humidity (weight=1), moderate air quality (weight=2), and no rainfall (weight=2), the blast exposure may be 18, which is the sum of the weights associated with all present factors.

In some embodiments of the invention, the sustained blast exposure may be a value enumerating the individual's exposure to all blast events within a single continuous period of time and may be the sum of all blast exposure values pertaining to blast events occurring within the single continuous period of time. For example, if an individual is exposed to three blast events in the space of a month, where the blast exposure associated with the first blast event is 15, the blast exposure associated with the second blast event is 12, and the blast exposure associated with the third blast event is 32, the sustained blast exposure for that month may be 59. In some embodiments of the invention, in calculating the sustained blast exposure, the blast exposure value associated with each blast event may be adjusted based on the period of time between blast events; for example, if a first blast event occurs greater than a threshold period of time prior to a second blast event, the exposure value of the first blast event may be minorly decreased proportionally to the period of time elapsed between blast events, to account for the dangerous effects on an individual of experiencing multiple blast events within a short period of time.

In some embodiments of the invention, the system may compare the blast exposure value or the sustained blast exposure against one or more threshold values, where each threshold value represents a level of blast exposure or sustained blast exposure that is associated with a different blast injury. Blast injuries may include traumatic brain injuries (TBI), injuries to the auditory system such as damage to the tympanic membrane or sound receptors within the cochlea and damage to blood vessels and neural pathways within the auditory system, injuries to the lungs and gastrointestinal tracts, et cetera. In some embodiments of the invention, a blast exposure or sustained blast exposure calculated exclusively from factors pertaining to the blast data may be compared against the one or more threshold values associated with different blast injuries. If the blast exposure or sustained blast exposure exceeds any of the threshold values, the system may identify a likelihood that the individual has sustained blast injuries corresponding with the threshold values. In some embodiments of the invention, the system may associate a specific blast injury with the presence of one or more combinations of factors, based on, for example, the input of an individual, medical professional, and/or identifying that specific blast injury as corresponding with one or more combinations of factors in historical data. For example, the system may associate a traumatic brain injury with scenarios where the individual was in an enclosed space and was either exposed to an overpressure wave exceeding a certain magnitude or was exposed to multiple smaller overpressure waves exceeding a certain frequency. In such embodiments, the system may identify a likelihood that the individual has sustained a particular blast injury if the one or more combinations of factors pertaining to that brain injury are present during the blast event.

In some embodiments of the invention, for example where the system has identified a likelihood of the individual experiencing a particular blast injury, the system may prompt the individual to seek medical attention, including, for example, a screening for that blast injury. The system may alert a medical professional of the individual's need for medical attention with respect to the particular blast injury. The system may prompt the individual and/or the medical professional via a graphical, text, and/or audio prompt including synthesized speech, digitized speech, or an alert sound via the client device associated with the individual and/or medical professional. In some embodiments of the invention, for example where the system has already identified a likelihood of the individual experiencing a particular blast injury, and/or where the individual has already been prompted and/or screened for the particular blast injury, the system may prompt the individual and/or medical professional an additional time to screen or re-screen for the blast injury where the blast exposure or sustained blast exposure has since increased by an amount corresponding to the threshold value associated with the blast injury.

In some embodiments of the invention, the blast exposure and any blast injury diagnoses made in connection with an individual, even where the blast injury diagnoses were not made as a direct result of the blast exposure or action by the system in connection with the blast exposure, may be recorded; climate factors, blast data, physical feature data, and position data associated with multiple recorded blast exposures and associated blast injury diagnoses may be analyzed to identify the effect of climate factors, physical features, position and movement, et cetera on blast injuries, and to improve (among other things) the accuracy of future calculations of blast exposure by adjusting the weights representing the importance of individual factors based on the historical effects. The system may adjust weights by, for example, comparing a number of different individuals during the same or similar blast events who experienced different blast injuries, and determining whether the weight already associated with the factors that differed between the individuals explain the difference in health outcomes; if not, the system may adjust the weight of one or more of the differing factors to better conform with the difference in health outcomes. From this process of analysis and adjustment, the system may yield important inferences regarding the effect of different factors on blast exposure, and may provide suggestions to the individual and/or medical professionals regarding factors, including climate factors, that mitigate or exacerbate the blast exposure and the harmful effects of a blast event; for example, the system may determine based on the weights that individuals who experience blast events during periods of high rainfall, poor air quality, and high wind speed have lower blast exposure, and are thereby less likely to experience a blast injury.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method, and program product to utilize a person's location, local climate data, and blast data during one or more blast events to assess blast exposure and diagnose a blast injury.

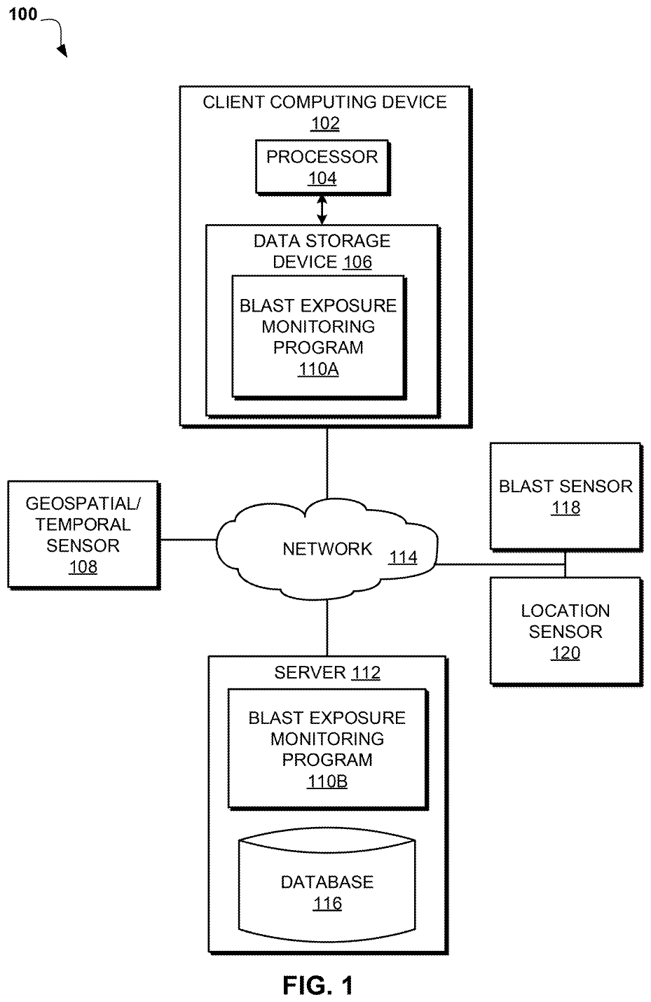

Referring to FIG. 1, an exemplary networked computer environment 100 is depicted, according to at least one embodiment. The networked computer environment 100 may include client computing device 102 and a server 112 interconnected via a communication network 114. According to at least one implementation, the networked computer environment 100 may include a plurality of client computing devices 102 and servers 112, of which only one of each is shown for illustrative brevity.

The communication network 114 may include various types of communication networks, such as a wide area network (WAN), local area network (LAN), a telecommunication network, a wireless network, a public switched network and/or a satellite network. The communication network 114 may include connections, such as wire, wireless communication links, or fiber optic cables. It may be appreciated that FIG. 1 provides only an illustration of one implementation and does not imply any limitations with regard to the environments in which different embodiments may be implemented. Many modifications to the depicted environments may be made based on design and implementation requirements.

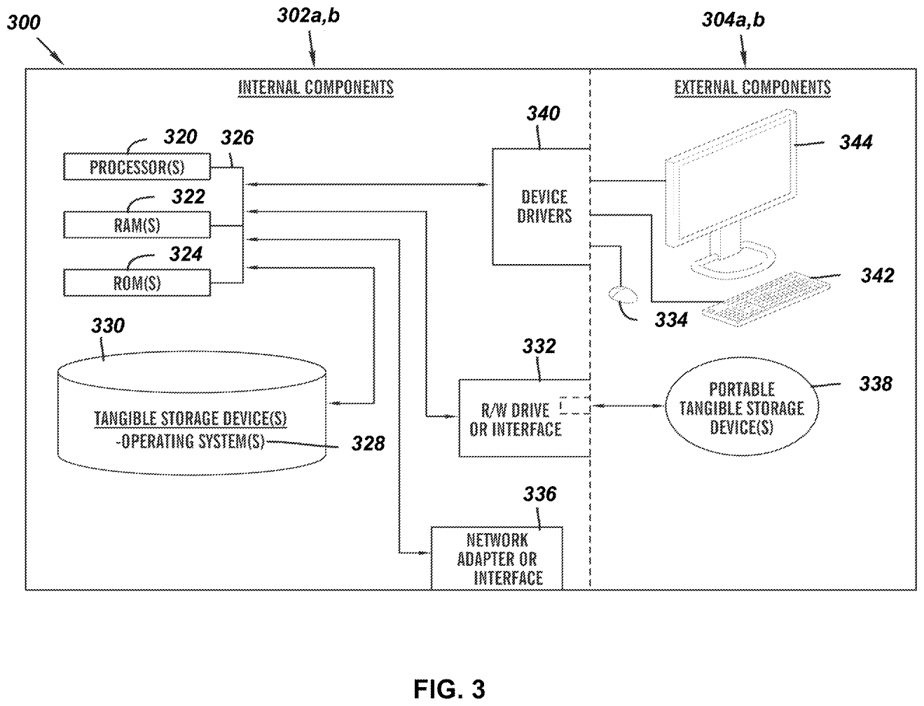

Client computing device 102 may include a processor 104 and a data storage device 106 that is enabled to host and run a blast exposure monitoring program 110A and communicate with the server 112 via the communication network 114, in accordance with one embodiment of the invention. Client computing device 102 may be, for example, a mobile device, a telephone, a personal digital assistant, a netbook, a laptop computer, a tablet computer, a desktop computer, or any type of computing device capable of running a program and accessing a network. As will be discussed with reference to FIG. 3, the client computing device 102 may include internal components 302a and external components 304a, respectively.

The server computer 112 may be a laptop computer, netbook computer, personal computer (PC), a desktop computer, or any programmable electronic device or any network of programmable electronic devices capable of hosting and running a blast exposure monitoring program 110B and a database 116 and communicating with the client computing device 102 via the communication network 114, in accordance with embodiments of the invention. As will be discussed with reference to FIG. 3, the server computer 112 may include internal components 302b and external components 304b, respectively. The server 112 may also operate in a cloud computing service model, such as Software as a Service (SaaS), Platform as a Service (PaaS), or Infrastructure as a Service (IaaS). The server 112 may also be located in a cloud computing deployment model, such as a private cloud, community cloud, public cloud, or hybrid cloud.

The geospatial/temporal sensor 108 may be any number or combination of sensors capable of gathering geospatial/temporal data from the environment of the individual during a blast event, such as barometers, windspeed indicators, rainfall gauges, weathervanes, thermometers et cetera; the geospatial/temporal data gathered by the geospatial/temporal sensors 108 may include any environmental factors or conditions that may have an effect on the propagation of pressure waves through the environment, such as temperature, wind speed, wind direction, humidity, air quality, rainfall, barometric pressure, et cetera. The geospatial/temporal sensor 108 may be operated by or otherwise in communication with blast exposure monitoring program 110A, 110B, for example through a direct link or through network 114.

The blast sensor 118 may be a pressure sensor capable of measuring the blast data at or near the person of the individual in real time or near-real-time. Blast data may be data pertaining to the pressure changes produced by a blast. As such, the blast sensor 118 may be a pressure sensor, capable of measuring potentially damaging overpressure values significantly above and beyond those of normal atmospheric pressure. The blast sensor 118 may be deployed on or near the person of the individual, for example incorporated into a wearable or otherwise portable mobile device worn or carried by the individual, installed in a vehicle belonging to the individual, et cetera. The blast sensor 118 may be operated by or otherwise in communication with blast exposure monitoring program 110A, 110B, for example through a direct link or through network 114.

The location sensor 120 may be a sensor capable of measuring the location of the individual during a blast event, in real time or near-real time. Location sensor 120 may be referred to herein as a GPS sensor, but one skilled in the art would understand location sensors 120 to be any hardware or software capable of locating the individual, for example, a near-field communications transceiver that gathers microlocation data associated with an individual by interfacing with the individual's mobile device, RFID tag reader, beacon, camera, motion detector, laser rangefinder, et cetera. In some embodiments of the invention, the location sensor 120 may be a software agent capable of deducing the geographical location of the individual's mobile device when it is connected to the internet, for example using the IP address, MAC address, et cetera. The location sensor 120 may be integrated into the individual's mobile device, and/or deployed within the environment of the individual and may be operated by or otherwise in communication with blast exposure monitoring program 110A, 110B, for example through a direct link or through network 114.

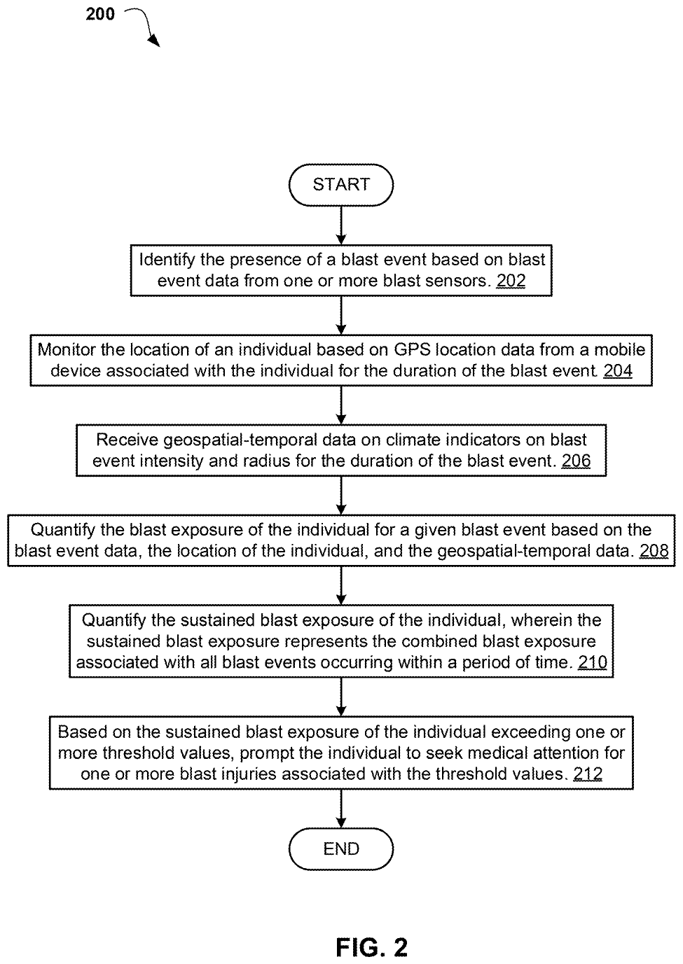

According to the present embodiment, the blast exposure monitoring program 110A, 110B may be a program enabled to utilize a person's location, local climate data, and blast data during one or more blast events to assess blast exposure and diagnose a blast injury. The blast exposure monitoring may be located on client computing device 102 or server 112 or on any other device located within network 114. Furthermore, blast exposure monitoring may be distributed in its operation over multiple devices, such as client computing device 102 and server 112. The blast exposure monitoring method is explained in further detail below with respect to FIG. 2.

Referring now to FIG. 2, an operational flowchart illustrating a blast exposure monitoring process 200 is depicted according to at least one embodiment. At 202, the blast exposure monitoring program 110A, 110B identifies the presence of a blast event based on blast event data from one or more blast sensors. The blast exposure monitoring program 110A, 110B may identify a presence and duration of a blast event, which may be determined in real time or near-real-time based on the blast data. The blast exposure monitoring program 110A, 110B may identify the presence of a blast event responsive to receiving a pressure wave of a magnitude that exceeds a threshold value, where the threshold value represents a pressure wave of a magnitude which is likely to have been caused by a blast event, and at or below which represents pressure fluctuations that are unlikely to have been caused by a blast event. In some embodiments of the invention, the blast exposure monitoring program 110A, 110B may identify the presence of a blast event where the magnitude and frequency of one or more pressure waves match a profile associated with an explosion. The blast exposure monitoring program 110A, 110B may determine when the blast event is over at a point where, subsequent to identifying the presence of a blast event, the blast sensors record no pressure waves exceeding the threshold value for greater than a threshold duration; the threshold duration may represent a maximum amount of time that may pass without pressure waves from a blast event before the blast event is likely to be over. The blast exposure monitoring program 110A, 110B may calculate the duration of the blast event by calculating an elapsed time between the blast exposure monitoring program 110A, 110B identifying the presence of the blast event and the blast exposure monitoring program 110A, 110B determining that the blast event is over.

At 204, blast exposure monitoring program 110A, 110B monitors the location of an individual based on GPS location data from a mobile device associated with the individual for the duration of the blast event. Responsive to blast exposure monitoring program 110A, 110B identifying the presence of a blast event, blast exposure monitoring program 110A, 110B may operate one or more location sensors 120 to collect location data on the individual until blast exposure monitoring program 110A, 110B determines that the blast event is over. In some embodiments of the invention, blast exposure monitoring program 110A, 110B may simply receive or acquire location data collected by one or more location sensors 120 from a web service, database, website, or any other data repository. The blast exposure monitoring program 110A, 110B may monitor the location of the individual in real time, and/or at intervals during the blast event, where the intervals may be regular points in time, for example every second or quarter second, at which the location of the individual is recorded.

At 206, blast exposure monitoring program 110A, 110B receives geospatial/temporal data on climate indicators on blast event intensity and radius for the duration of the blast event. Responsive to blast exposure monitoring program 110A, 110B identifying the presence of a blast event, blast exposure monitoring program 110A, 110B may operate one or more geospatial/temporal sensors 108 to collect data on any environmental factors or conditions in the area of the blast event that may have an effect on the propagation of pressure waves through the environment, including temperature, wind speed, wind direction, humidity, air quality, rainfall, barometric pressure, et cetera, until blast exposure monitoring program 110A, 110B determines that the blast event is over. In some embodiments of the invention, blast exposure monitoring program 110A, 110B may receive or acquire geospatial/temporal data during or subsequent to the blast event from a database, online service, website, or other form of online or data repository, where the received or acquired geospatial/temporal data was collected during the blast event and in the same or a nearby location to the individual and/or the blast.

At 208, blast exposure monitoring program 110A, 110B quantifies the blast exposure of the individual for a given blast event based on the blast event data, the location of the individual, and the geospatial/temporal data. The blast exposure for an individual may be a representation of the individual's exposure to a given blast event at given intervals of time, for example every second, or every half a second, during the blast event, and may comprise all features or information of the blast, location of the individual, environmental conditions, and/or surrounding terrain features necessary to assess the individual's exposure to the blast and likelihood of sustaining a blast injury. The blast exposure may include all pressure waves experienced by the individual over the course of a blast event, including the peak of the initial pressure wave, the duration of the overpressure, the time from the incident blast wave, and the frequency of the waves. The blast exposure may further include the type of pressure waves experienced by the individual, including complex waves, reflective waves, Friedlander waves, et cetera. The blast exposure monitoring program 110A, 110B may quantify the blast exposure by assigning weights to each of a number of factors drawn from the geospatial/temporal data, blast data, and/or location data, where the weight may represent the relative amount to which the factor contributes to the blast exposure and/or likelihood of blast injury. The blast exposure monitoring program 110A, 110B may dynamically adjust the weight associated with any given factor based on new data, to more precisely represent the amount to which the factor contributes to the likelihood of blast injury. The blast exposure monitoring program 110A, 110B may then sum the weight of all factors present during the blast event to produce a single value which comprises the blast exposure. In some embodiments of the invention, blast exposure monitoring program 110A, 110B may sum the weight of all factors present during each interval of time comprising the blast event to produce a value for each interval which comprises the blast exposure at that interval; the blast exposure monitoring program 110A, 110B may then average or sum the blast values at each interval to produce a single value representing the blast exposure associated with the blast event.

In some embodiments of the invention, the blast exposure monitoring program 110A, 110B may determine the location of the blast based on the location of the individual and the blast data during the duration of the blast event. In some embodiments, the blast exposure monitoring program 110A, 110B may receive the location of the blast, for example from a system designed to triangulate or otherwise locate a blast. In some embodiments of the invention, for example where blast data and location data are being recorded for a plurality of individuals, the blast exposure monitoring program 110A, 110B may locate the blast based on the location and recorded pressure waves associated with a plurality of individuals at the same interval of time and/or during the same blast event.

In some embodiments of the invention, the blast exposure monitoring program 110A, 110B may calculate blast exposure by taking into account factors comprising physical features such as structures, hills, rocks, et cetera near the individual which may offer shelter from the blast. The blast exposure monitoring program 110A, 110B may analyze maps of the terrain near the individual to determine, for example using object detection methods, topographical data, geological surveys, et cetera, what physical features such as structures, geological/topographical features, objects, et cetera were near the individual. In some embodiments of the invention, the blast exposure monitoring program 110A, 110B may determine the strength, density, pressure conduction, material composition, flexibility, or other properties of one or more physical features, for example by looking up physical properties of objects identified using object detection methods, determining the type and composition of rocks and terrain features based on geological data, et cetera. In some embodiments of the invention, for example where the blast exposure monitoring program 110A, 110B identifies the location of the blast, the blast exposure monitoring program 110A, 110B may identify all or some subset of physical features between the individual and the location of the blast at any point during the duration of the blast event, such that the physical features may have shielded the individual or disrupted the pressure waves caused by the blast event prior to the pressure waves reaching the individual. The blast exposure monitoring program 110A, 110B may consider identified physical features as factors, and may weight each factor based on, for example, an extent to which the physical feature may shield the individual from pressure waves, which in turn may be based on the physical feature's location relative to the individual and/or the blast, and the size, strength, and/or material composition of the physical feature.

At 210, blast exposure monitoring program 110A, 110B quantifies the sustained blast exposure of the individual, wherein the sustained blast exposure represents the combined blast exposure associated with all blast events occurring within a period of time. The period of time may be a single continuous period of time, such as a month or year. The blast exposure monitoring program 110A, 110B may quantify the sustained blast exposure by calculating the sum of all blast exposure values pertaining to blast events occurring within the period of time.

At 212, blast exposure monitoring program 110A, 110B, based on the sustained blast exposure of the individual exceeding one or more threshold values, prompts the individual to seek medical attention for one or more blast injuries associated with the threshold values. The blast exposure monitoring program 110A, 110B may compare the sustained blast exposure against one or more threshold values, where each threshold value represents a level of sustained blast exposure that is associated with a different blast injury. Blast injuries may include traumatic brain injuries (TBI), injuries to the auditory system such as damage to the tympanic membrane or sound receptors within the cochlea and damage to blood vessels and neural pathways within the auditory system, injuries to the lungs and gastrointestinal tracts, et cetera. In some embodiments of the invention, blast exposure monitoring program 110A, 110B may calculate a sustained blast exposure exclusively from factors pertaining to the blast data and may compare this sustained blast exposure value against the one or more threshold values associated with different blast injuries. If the sustained blast exposure exceeds any of the threshold values, the blast exposure monitoring program 110A, 110B may identify a likelihood that the individual has sustained blast injuries corresponding with the threshold values.

In some embodiments of the invention, the blast exposure monitoring program 110A, 110B may associate a specific blast injury with the presence of one or more combinations of factors, based on, for example, the input of an individual, medical professional, and/or identifying that specific blast injury as corresponding with one or more combinations of factors in historical data. For example, the blast exposure monitoring program 110A, 110B may associate a traumatic brain injury with scenarios where the individual was in an enclosed space and was either exposed to an overpressure wave exceeding a certain magnitude or was exposed to multiple smaller overpressure waves exceeding a certain frequency. In such embodiments, the blast exposure monitoring program 110A, 110B may identify a likelihood that the individual has sustained a particular blast injury if the one or more combinations of factors pertaining to that brain injury are present during the blast event.

In some embodiments of the invention, for example where the blast exposure monitoring program 110A, 110B has identified a likelihood of the individual experiencing a particular blast injury, the blast exposure monitoring program 110A, 110B may prompt the individual to seek medical attention, including, for example, a screening for that blast injury. The blast exposure monitoring program 110A, 110B may alert a medical professional of the individual's need for medical attention with respect to the particular blast injury. The blast exposure monitoring program 110A, 110B may prompt the individual and/or the medical professional via a graphical, text, and/or audio prompt including synthesized speech, digitized speech, or an alert sound via the client device associated with the individual and/or medical professional. In some embodiments of the invention, for example where the blast exposure monitoring program 110A, 110B has already identified a likelihood of the individual experiencing a particular blast injury, and/or where the individual has already been prompted and/or screened for the particular blast injury, the blast exposure monitoring program 110A, 110B may prompt the individual and/or medical professional an additional time to screen or re-screen for the blast injury where the blast exposure or sustained blast exposure has since increased by an amount corresponding to the threshold value associated with the blast injury.

It may be appreciated that FIG. 2 provides only an illustration of one implementation and does not imply any limitations with regard to how different embodiments may be implemented. Many modifications to the depicted environments may be made based on design and implementation requirements. For example, prompting the individual to seek medical attention for one or more blast injuries associated with the threshold values may be based on the blast exposure of the individual exceeding the one or more threshold values, instead of the sustained blast exposure.

FIG. 3 is a block diagram 300 of internal and external components of the client computing device 102 and the server 112 depicted in FIG. 1 in accordance with an embodiment of the present invention. It should be appreciated that FIG. 3 provides only an illustration of one implementation and does not imply any limitations with regard to the environments in which different embodiments may be implemented. Many modifications to the depicted environments may be made based on design and implementation requirements.

The data processing system 302, 304 is representative of any electronic device capable of executing machine-readable program instructions. The data processing system 302, 304 may be representative of a smart phone, a computer system, PDA, or other electronic devices. Examples of computing systems, environments, and/or configurations that may represented by the data processing system 302, 304 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

The client computing device 102 and the server 112 may include respective sets of internal components 302 a, b and external components 304 a, b illustrated in FIG. 3. Each of the sets of internal components 302 include one or more processors 320, one or more computer-readable RAMs 322, and one or more computer-readable ROMs 324 on one or more buses 326, and one or more operating systems 328 and one or more computer-readable tangible storage devices 330. The one or more operating systems 328, the blast exposure monitoring program 110A in the client computing device 102, and the blast exposure monitoring program 110B in the server 112 are stored on one or more of the respective computer-readable tangible storage devices 330 for execution by one or more of the respective processors 320 via one or more of the respective RAMs 322 (which typically include cache memory). In the embodiment illustrated in FIG. 3, each of the computer-readable tangible storage devices 330 is a magnetic disk storage device of an internal hard drive. Alternatively, each of the computer-readable tangible storage devices 330 is a semiconductor storage device such as ROM 324, EPROM, flash memory or any other computer-readable tangible storage device that can store a computer program and digital information.

Each set of internal components 302 a, b also includes a R/W drive or interface 332 to read from and write to one or more portable computer-readable tangible storage devices 338 such as a CD-ROM, DVD, memory stick, magnetic tape, magnetic disk, optical disk, or semiconductor storage device. A software program, such as the blast exposure monitoring program 110A, 110B, can be stored on one or more of the respective portable computer-readable tangible storage devices 338, read via the respective R/W drive or interface 332, and loaded into the respective hard drive 330.

Each set of internal components 302 a, b also includes network adapters or interfaces 336 such as a TCP/IP adapter cards, wireless Wi-Fi interface cards, or 3G or 4G wireless interface cards or other wired or wireless communication links. The blast exposure monitoring program 110A in the client computing device 102 and the blast exposure monitoring program 110B in the server 112 can be downloaded to the client computing device 102 and the server 112 from an external computer via a network (for example, the Internet, a local area network or other, wide area network) and respective network adapters or interfaces 336. From the network adapters or interfaces 336, the blast exposure monitoring program 110A in the client computing device 102 and the blast exposure monitoring program 110B in the server 112 are loaded into the respective hard drive 330. The network may comprise copper wires, optical fibers, wireless transmission, routers, firewalls, switches, gateway computers and/or edge servers.

Each of the sets of external components 304 a, b can include a computer display monitor 344, a keyboard 342, and a computer mouse 334. External components 304 a, b can also include touch screens, virtual keyboards, touch pads, pointing devices, and other human interface devices. Each of the sets of internal components 302 a, b also includes device drivers 340 to interface to computer display monitor 344, keyboard 342, and computer mouse 334. The device drivers 340, R/W drive or interface 332, and network adapter or interface 336 comprise hardware and software (stored in storage device 330 and/or ROM 324).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

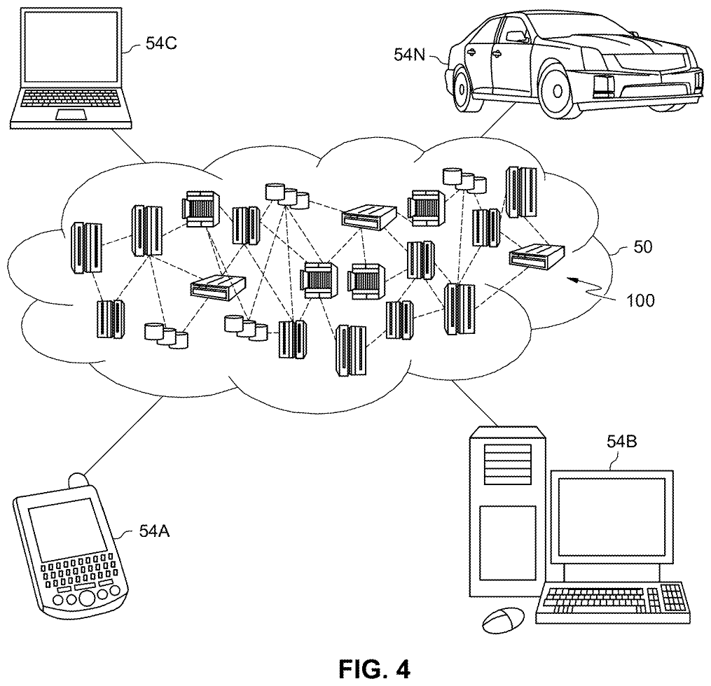

Referring now to FIG. 4, illustrative cloud computing environment 50 is depicted. As shown, cloud computing environment 50 comprises one or more cloud computing nodes 100 with which local computing devices used by cloud consumers, such as, for example, personal digital assistant (PDA) or cellular telephone 54A, desktop computer 54B, laptop computer 54C, and/or automobile computer system 54N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 100 and cloud computing environment 50 can communicate with any type of computerized device over any type of network and/or network addressable connection (e.g., using a web browser).

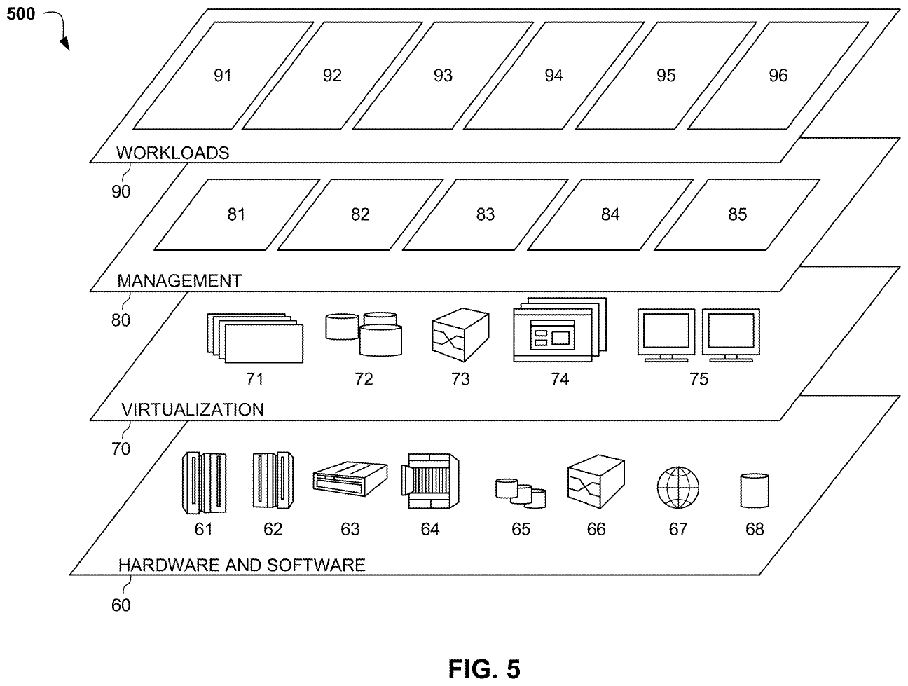

Referring now to FIG. 5, a set of functional abstraction layers 500 provided by cloud computing environment 50 is shown. It should be understood in advance that the components, layers, and functions shown in FIG. 5 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture-based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below.

Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and blast exposure monitoring 96. The blast exposure monitoring 96 may be enabled to utilize a person's location, local climate data, and blast data during one or more blast events to assess blast exposure and diagnose a blast injury.

The descriptions of the various embodiments of the present invention have been presented for purposes of illustration but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A processor-implemented method for diagnosing one or more blast injuries of an individual based on sustained blast exposure, the method comprising:
responsive to identifying the presence of a blast event, monitoring the location of an individual for the duration of the blast event;
based on the monitored location, a plurality of geospatial/temporal data corresponding with a time and a location of the blast event, and a plurality of blast data corresponding with the blast event, determining a blast exposure associated with the blast event for the individual;
determining a sustained blast exposure of the individual comprising the blast exposures associated with a plurality of blast events during a single, continuous period of time; and
responsive to the sustained blast exposure of the individual exceeding one or more threshold values, prompting the individual to seek medical attention for one or more blast injuries associated with the one or more threshold values.

2. The method of claim 1, further comprising:
responsive to the sustained blast exposure of the individual increasing by a threshold value, prompting the individual an additional time to seek medical attention for the one or more blast injuries associated with the threshold value.

3. The method of claim 1, wherein the blast exposure is based on a size, shape, strength, or position of one or more physical features near the individual.

4. The method of claim 1, wherein the blast exposure is calculated for each of a plurality of intervals during the blast event.

5. The method of claim 1, further comprising:
identifying, based on a plurality of weights associated with a plurality of factors comprising the blast exposure, an effect of one or more climate factors on the blast exposure of the blast event.

6. The method of claim 1, further comprising:
identifying, based on a plurality of weights associated with a plurality of factors comprising the blast exposure, a plurality of factors that mitigate or exacerbate the blast exposure of the blast event.

7. The method of claim 1, further comprising:
responsive to the blast exposure of the individual exceeding one or more threshold values, prompting the individual to seek medical attention for one or more blast injuries associated with the one or more threshold values.

8. A computer system for diagnosing one or more blast injuries of an individual based on sustained blast exposure, the computer system comprising:
one or more blast sensors, one or more location sensors, one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
responsive to identifying the presence of a blast event, monitoring the location of an individual for the duration of the blast event;
based on the monitored location, a plurality of geospatial/temporal data corresponding with a time and a location of the blast event, and a plurality of blast data corresponding with the blast event, determining a blast exposure associated with the blast event for the individual;
determining a sustained blast exposure of the individual comprising the blast exposures associated with a plurality of blast events during a single, continuous period of time; and
responsive to the sustained blast exposure of the individual exceeding one or more threshold values, prompting the individual to seek medical attention for one or more blast injuries associated with the one or more threshold values.

9. The computer system of claim 8, further comprising:
responsive to the sustained blast exposure of the individual increasing by a threshold value, prompting the individual an additional time to seek medical attention for the one or more blast injuries associated with the threshold value.

10. The computer system of claim 8, wherein the blast exposure is based on a size, shape, strength, or position of one or more physical features near the individual.

11. The computer system of claim 8, wherein the blast exposure is calculated for each of a plurality of intervals during the blast event.

12. The computer system of claim 8, further comprising:
identifying, based on a plurality of weights associated with a plurality of factors comprising the blast exposure, an effect of one or more climate factors on the blast exposure of the blast event.

13. The computer system of claim 8, further comprising:
identifying, based on a plurality of weights associated with a plurality of factors comprising the blast exposure, a plurality of factors that mitigate or exacerbate the blast exposure of the blast event.

14. The computer system of claim 8, further comprising:
responsive to the blast exposure of the individual exceeding one or more threshold values, prompting the individual to seek medical attention for one or more blast injuries associated with the one or more threshold values.

15. A computer program product for diagnosing one or more blast injuries of an individual based on sustained blast exposure, the computer program product comprising:
one or more computer-readable tangible storage medium and program instructions stored on at least one of the one or more tangible storage medium, the program instructions executable by a processor to cause the processor to perform a method comprising:
responsive to identifying the presence of a blast event, monitoring the location of an individual for the duration of the blast event;
based on the monitored location, a plurality of geospatial/temporal data corresponding with a time and a location of the blast event, and a plurality of blast data corresponding with the blast event, determining a blast exposure associated with the blast event for the individual;
determining a sustained blast exposure of the individual comprising the blast exposures associated with a plurality of blast events during a single, continuous period of time; and
responsive to the sustained blast exposure of the individual exceeding one or more threshold values, prompting the individual to seek medical attention for one or more blast injuries associated with the one or more threshold values.

16. The computer program product of claim 15, further comprising:
responsive to the sustained blast exposure of the individual increasing by a threshold value, prompting the individual an additional time to seek medical attention for the one or more blast injuries associated with the threshold value.

17. The computer program product of claim 15, wherein the blast exposure is based on a size, shape, strength, or position of one or more physical features near the individual.

18. The computer program product of claim 15, wherein the blast exposure is calculated for each of a plurality of intervals during the blast event.

19. The computer program product of claim 15, further comprising:
identifying, based on a plurality of weights associated with a plurality of factors comprising the blast exposure, an effect of one or more climate factors on the blast exposure of the blast event.

20. The computer program product of claim 15, further comprising:
identifying, based on a plurality of weights associated with a plurality of factors comprising the blast exposure, a plurality of factors that mitigate or exacerbate the blast exposure of the blast event.
